(19) United States
(12) Patent Application Publication
(10) Pub. No.: US 2025/0361163 A1
(43) Pub. Date: Nov. 27, 2025

(54) INLINE WATER MONITORING SYSTEM

(71) Applicant: ZODIAC POOL SYSTEMS LLC, Carlsbad, CA (US)

(72) Inventors: Timothy McNeil, Carlsbad, CA (US); Anthony Levacque, Carlsbad, CA (US); Mark Bauckman, San Marcos, CA (US); Hendrikus Johannes van der Meijden, Glen Austin (ZA); Abian van der Meijden, Glen Austin (ZA)

(73) Assignee: ZODIAC POOL SYSTEMS LLC, Carlsbad, CA (US)

(21) Appl. No.: 19/213,603

(22) Filed: May 20, 2025

Related U.S. Application Data
(60) Provisional application No. 63/651,719, filed on May 24, 2024.

(51) Int. Cl. C02F 1/68; C02F 103/42; E04H 4/12

(52) U.S. Cl. CPC C02F 1/686; E04H 4/1281; C02F 2103/42; C02F 2201/005; C02F 2209/001

(57) ABSTRACT

A water quality monitoring system for a swimming pool or spa is provided inline with other systems of the swimming pool or spa. The water quality monitoring system may include a double plunger, a testing chamber with a fixed volume, and a multiport rotary valve. A method of monitoring water quality of water of a swimming pool or spa may include drawing water into the testing chamber using the double plunger, rotating the multiport rotary valve to first position enabling fluid communication with a first reagent, drawing the first reagent into the testing chamber using the double plunger, mixing the first reagent and the water in the testing chamber using the double plunger, and measuring a water parameter of the mixed water and the first reagent in the testing chamber using a photometer.

### REFERENCE TO RELATED APPLICATION

This application claims the benefit of and priority to U.S. Provisional Patent Application No. 63/651,719, filed on May 24, 2024, and entitled INLINE WATER MONITORING SYSTEM, the content of which is hereby incorporated by reference in its entirety.

### FIELD OF THE INVENTION

The invention relates to water testing or monitoring systems, and more particularly, but not necessarily exclusively, to water testing or monitoring systems for swimming pools or spas.

### BACKGROUND

Maintaining water quality is important for swimming pools, spas, hot tubs, and other water containing vessels (hereinafter “swimming pools or spas”) to avoid issues for users of the swimming pool or spa as well as equipment of the swimming pool or spa. For example, if the water chemistry of the swimming pool or spa is off, a health hazard may be posed to users and/or operation of various pool equipment and/or systems may be compromised.

Conventional user-controlled approaches for monitoring water quality include chemistry kits, remote testing, and maintenance service calls. Conventional chemistry kits may be complicated and not user-friendly to pool owners, and the individual using the chemistry kit may be uncertain of the results, Remote testing requires taking a sample of water to a store or chemistry laboratory to analyze the sample but may pose issues of sample contamination, improper storage, and/or a change in water chemistry during transit. Service calls rely on the availability of maintenance personnel and further rely on the expertise of the person to properly conduct water quality testing and to properly understand and interpret the results from such testing. As such, many existing approaches to monitoring water quality may be inaccurate, inconvenient, labor-intensive and time-consuming.

In addition to user-controlled approaches, other approaches for monitoring water quality have included probes and floating devices, but these other approaches also suffer from various deficiencies. For example, probes may drift and age over time, often requiring extensive maintenance for cleaning and calibration of the probes. In addition, it may be difficult to predict or troubleshoot an incorrect measurement from a probe, and the incorrect measurement may lead to over-dosing or under-dosing of chemicals into the pool. Floating devices may become stuck in non-desirable locations and may be removed from the pool by any user, thereby providing incorrect measurements or compromising the performance of the device. In addition, floating devices are battery-operated, thereby requiring periodic maintenance to ensure proper powering of such devices.

U.S. Pat. No. 7,988,916 to Bremauer (“Bremauer”) describes “an apparatus for precisely mixing and/or analysing small volumes of fluids for optic values.” Bremauer at col. 1, 11. 8-10. According to Bremauer, the “apparatus for measuring a range of small volumes” includes “a) a single reaction chamber, b) a reciprocatable piston in said chamber, c) a first inlet to said chamber . . . d) at least one further inlet . . . e) a sealable outlet . . . f) said piston is operable within said chamber to selectively and precisely vary the internal volume of said chamber . . . g) said first inlet has a first valve selectively operable . . . until a predetermined volume of said first fluid is drawn into said chamber . . . h) said second inlet has a second valve selectively operable . . . to progressively draw said second fluid into said chamber until a predetermined condition is met . . . [and] j) said outlet is sealed by an outlet valve located in said base adjacent the internal surface of said chamber.” Bremauer at col. 3, 11. 33-67.

### SUMMARY

The terms “invention,” “the invention,” “this invention” and “the present invention” used in this patent are intended to refer broadly to all of the subject matter of this patent and the patent claims below. Statements containing these terms should be understood not to limit the subject matter described herein or to limit the meaning or scope of the patent claims below. Embodiments of the invention covered by this patent are defined by the claims below, not this summary. This summary is a high-level overview of various embodiments of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification of this patent, any or all drawings, and each claim.

According to certain embodiments, a method of monitoring water quality of water of a swimming pool or spa includes mixing the water and a reagent in a fixed volume using a double plunger.

According to some embodiments, a system for monitoring water quality of a swimming pool or spa includes a double plunger and a fixed volume, and the double plunger is configured to mix water of the swimming pool or spa and a reagent within the fixed volume.

According to certain embodiments, a system for monitoring water quality of a swimming pool or spa includes a double plunger and a multiport rotary valve.

According to various embodiments, a reagent based testing system includes ceramic valves for controlling water and reagent flow.

According to some embodiments, a system for monitoring water quality of a swimming pool or spa includes a double plunger and a multiport ceramic valve.

According to certain embodiments, a system for monitoring water quality of a swimming pool or spa is configured to measure water parameters using liquid reagents and a photometer and includes a single valve for controlling a flow of water and the liquid reagents into a testing chamber.

According to some embodiments, a method of monitoring water quality of water of a swimming pool or spa includes receiving a flow of water from a circulation system of the swimming pool or spa and measuring one or more water parameters using liquid reagents and a photometer.

According to various embodiments, a system for monitoring water quality of a swimming pool or spa includes a multiport rotary valve with at least a first reagent dispensing port for a first reagent and a second reagent dispensing port for a second reagent. In certain embodiments, the first reagent dispensing port and the second reagent dispensing port are different distances from a center of the multiport rotary valve.

According to some embodiments, a system for monitoring water quality of a swimming pool or spa includes a multiport rotary valve assembly, and the multiport rotary valve assembly comprises a plurality of reagent inflow ports and a plurality of reagent dispensing ports. In certain embodiments, the plurality of reagent inflow ports are a same distance from a center of the multiport rotary valve assembly, and at least a first reagent dispensing port and a second reagent dispensing port of the plurality of reagent dispensing ports are different distances from the center of the multiport rotary valve assembly.

According to some embodiments, a system for monitoring water quality of a swimming pool or spa includes a double plunger, a testing chamber having a fixed volume, and a multiport rotary valve.

According to various embodiments, a system for monitoring water quality of a swimming pool or spa includes a testing cell defining a testing chamber and a single valve for controlling a flow of water and the liquid reagents into a testing chamber and for controlling a flow of water and liquid reagent from testing chamber.

According to certain embodiments, a method of monitoring water quality of water of a swimming pool or spa includes (i) drawing water into a testing chamber using a double plunger, (ii) rotating a multiport rotary valve to first position enabling fluid communication with a first reagent, (iii) drawing the first reagent into the testing chamber using the double plunger, (iv) mixing the first reagent and the water in the testing chamber using the double plunge, and (v) measuring a water parameter of the mixed water and the first reagent in the testing chamber using a photometer.

Various implementations described in the present disclosure can include additional systems, methods, features, and advantages, which cannot necessarily be expressly disclosed herein but will be apparent to one of ordinary skill in the art upon examination of the following detailed description and accompanying drawings. It is intended that all such systems, methods, features, and advantages be included within the present disclosure and protected by the accompanying claims.

### BRIEF DESCRIPTION OF THE DRAWINGS

The features and components of the following figures are illustrated to emphasize the general principles of the present disclosure. Corresponding features and components throughout the figures can be designated by matching reference characters for the sake of consistency and clarity.

FIG. 1 illustrates a pool system according to embodiments.

FIG. 2 is a perspective view a monitoring system of the pool system of FIG. 1 according to embodiments.

FIG. 3 is a side view of the monitoring system of FIG. 2 according to embodiments.

FIG. 4 is a sectional view of the monitoring system of FIG. 2 according to embodiments.

FIG. 5 is a sectional view of a portion of the monitoring system of FIG. 2 according to embodiments.

FIG. 6 is a perspective view of a monitoring device of the monitoring system of FIG. 2 according to embodiments.

FIG. 7 is another perspective view of the monitoring device of FIG. 6 according to embodiments.

FIG. 8 is a perspective view of an interface assembly of the monitoring device of FIG. 6 according to embodiments.

FIG. 9 is an exploded view of the interface assembly of FIG. 8 according to embodiments.

FIG. 10 is a top view of the interface assembly of FIG. 8 according to embodiments.

FIG. 11 is a bottom view of the interface assembly of FIG. 8 according to embodiments.

FIG. 12 is a top view of a valve gasket of the interface assembly of FIG. 8 according to embodiments.

FIG. 13 is a top view of an upper plate of the interface assembly of FIG. 8 according to embodiments.

FIG. 14 is a bottom view of the upper plate of FIG. 13 according to embodiments.

FIG. 15 is a top view of a lower plate of the interface assembly of FIG. 8 according to embodiments.

FIG. 16 is a sectional view of the lower plate of FIG. 15 according to embodiments.

FIG. 17 is a bottom view of the lower plate of FIG. 15 according to embodiments.

FIG. 18 is a top view of the monitoring device of FIG. 6 according to embodiments.

FIG. 19 is another top view of the monitoring device of FIG. 6 according to embodiments.

FIG. 20 is another top view of the monitoring device of FIG. 6 according to embodiments.

FIG. 21 is another top view of the monitoring device of FIG. 6 according to embodiments,

FIG. 22 is a perspective view of the monitoring device of FIG. 6 according to embodiments,

FIG. 23 is another top view of the monitoring device of FIG. 6 according to embodiments.

FIG. 24 is a sectional view of a portion of the monitoring device of FIG. 6 according to embodiments.

FIG. 25 is a sectional view of a portion of the monitoring device of FIG. 6 according to embodiments.

FIG. 26 is a sectional view of a portion of the monitoring device of FIG. 6 according to embodiments.

FIG. 27 is a sectional view of a portion of the monitoring device of FIG. 6 according to embodiments.

FIG. 28 is a sectional view of a cartridge assembly of the monitoring system of FIG. 2 according to embodiments,

FIG. 29 is another sectional view of the cartridge assembly of FIG. 28 according to embodiments,

FIG. 30 is a perspective view of a portion of the cartridge assembly of FIG. 28 according to embodiments.

FIG. 31 is a side view of a portion of the cartridge assembly of FIG. 28 according to embodiments.

FIG. 32 is a top view of a portion of the cartridge assembly of FIG. 28 according to embodiments.

FIG. 33 is a perspective view of a valve plate of the cartridge assembly of FIG. 28 according to embodiments.

FIG. 34 is a sectional view of a portion of the monitoring system of FIG. 2 according to embodiments.

FIG. 35 is a perspective view of a monitoring system for the pool system of FIG. 1 according to embodiments.

FIG. 36 is another perspective view of the monitoring system of FIG. 35 according to embodiments,

FIG. 37 is a sectional view of the monitoring system of FIG. 35 according to embodiments.

FIG. 38 is another sectional view of the monitoring system of FIG. 35 according to embodiments.

FIG. 39 is a sectional view of a portion of the monitoring system of FIG. 35 according to embodiments.

FIG. 40 is a sectional view of a portion of the monitoring system of FIG. 35 according to embodiments.

FIG. 41 is a sectional view of a portion of the monitoring system of FIG. 35 according to embodiments.

FIG. 42 is a sectional view of a portion of the monitoring system of FIG. 35 according to embodiments,

FIG. 43 is a sectional view of a portion of the monitoring system of FIG. 35 according to embodiments.

FIG. 44 is a sectional view of a portion of the monitoring system of FIG. 35 according to embodiments.

FIG. 45 is a sectional view of a portion of the monitoring system of FIG. 35 according to embodiments,

FIG. 46 is a sectional view of a portion of the monitoring system of FIG. 35 according to embodiments.

FIG. 47 is a sectional view of a portion of the monitoring system of FIG. 35 according to embodiments.

### DESCRIPTION OF THE INVENTION

Described herein are systems and methods for monitoring water of a swimming pool or spa. In certain embodiments, the systems and methods described herein may provide automatic, accurate, and reliable measurement of water parameters in a pool system, such as locally to the pool system. The measurements may be provided to a user via a user interface of the system itself, a user interface of a swimming pool or spa system, and/or remotely to a user device as desired (e.g., via an application running on the user device).

In certain embodiments, the systems and methods described herein may provide a monitoring system for measuring one or more water parameters which is inline with other systems of the swimming pool or spa, such as but not limited to a water circulation system. In some embodiments, the systems and methods described herein may provide automatic measurement of a plurality of water parameters using a plurality of reagents while minimizing and/or eliminating cross-contamination of reagents during a measurement cycle.

In various embodiments, the systems and methods described herein may mix water and liquid reagent in a fixed volume, which may provide improved accuracy and reliability of measurements performed on the mixed water and reagent. In certain embodiments, the systems and methods described herein may utilize a dual plunger for drawing water, drawing a reagent, and mixing the water and reagent. In some embodiments, the systems and methods described herein may provide an inline monitoring system for measuring one or more water parameters using liquid reagent and a photometer.

Compared to traditional approaches, the systems and methods described herein may eliminate any need for a user to perform testing manually or physically. Various other benefits and advantages may be realized with the systems, devices, and methods provided herein, and the aforementioned advantages should not be considered limiting.

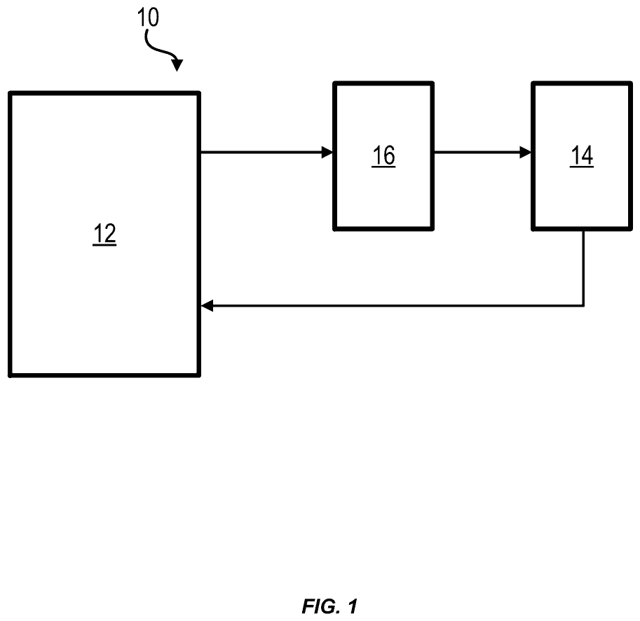

FIG. 1 illustrates an example of a pool system 10 according to embodiments. The pool system 10 generally includes a swimming pool or spa 12 (hereinafter “pool 12”) and a monitoring system 14 for measuring one or more water parameters of water of the pool 12. As non-limiting examples and as discussed in detail below, the monitoring system 14 may measure water parameters such as but not limited to pH, free chlorine, total chlorine, total alkalinity, cyanuric acid concentration, and/or other parameters. In some embodiments, the monitoring system 14 may measure one water parameter, although in other embodiments, the monitoring system 14 may measure a plurality of water parameters, such as two water parameters, three water parameters, four water parameters, five water parameters, and/or more than five water parameters. As discussed in detail below, the monitoring system 14 may include means for drawing and mixing water and a reagent within a testing chamber and means for measuring a water parameter of the mixed water within the testing chamber.

In certain embodiments and compared to traditional approaches, the monitoring system 14 may be provided inline with one or more systems or equipment of the pool system 10. As an example, and as illustrated in FIG. 1, the monitoring system 14 may be provided inline with a pump 16 of a water circulation system of the pool system 10. In other embodiments, the monitoring system 14 may be provided inline at other locations and/or relative to the pump 16 and/or other equipment of the pool system 10 as desired. As non-limiting examples, the monitoring system 14 may be provided inline to one or more of a heater, a chemical dosing (or sanitation) system, a filter, a skimmer, combinations thereof, and/or other equipment or combinations thereof as desired.

### Monitoring System

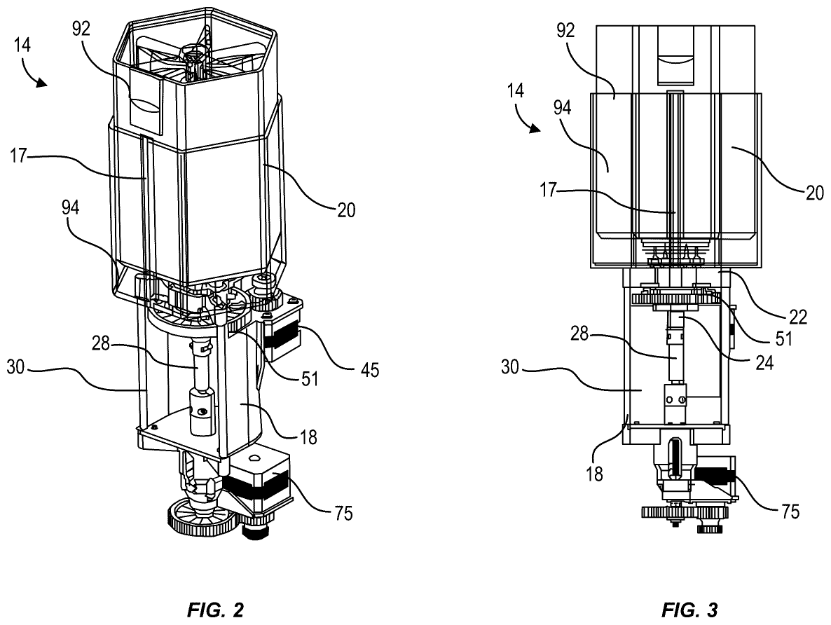

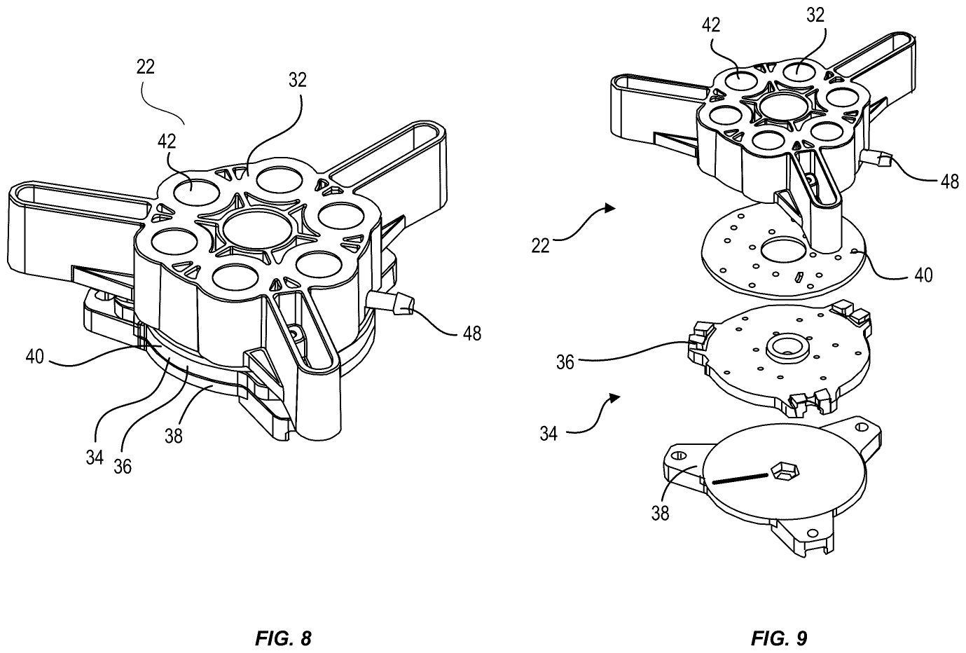

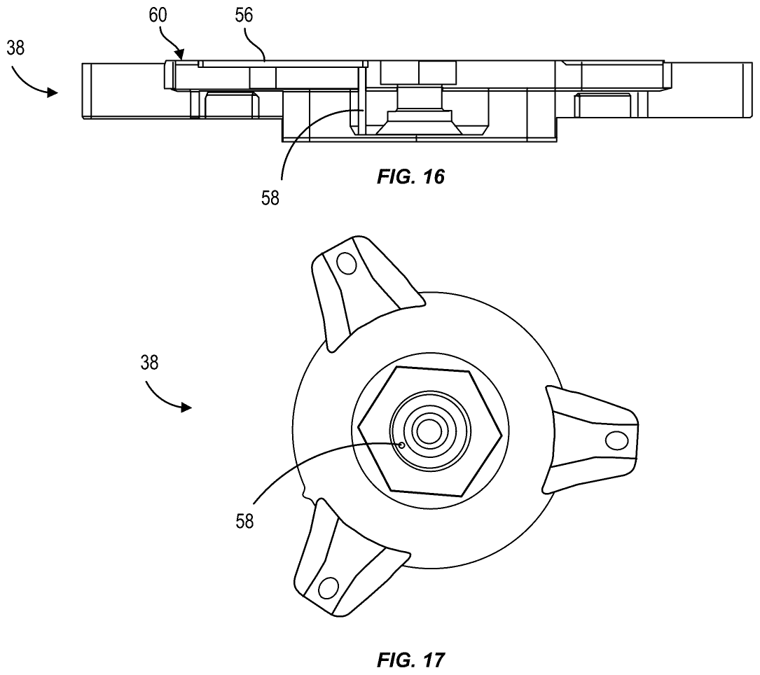

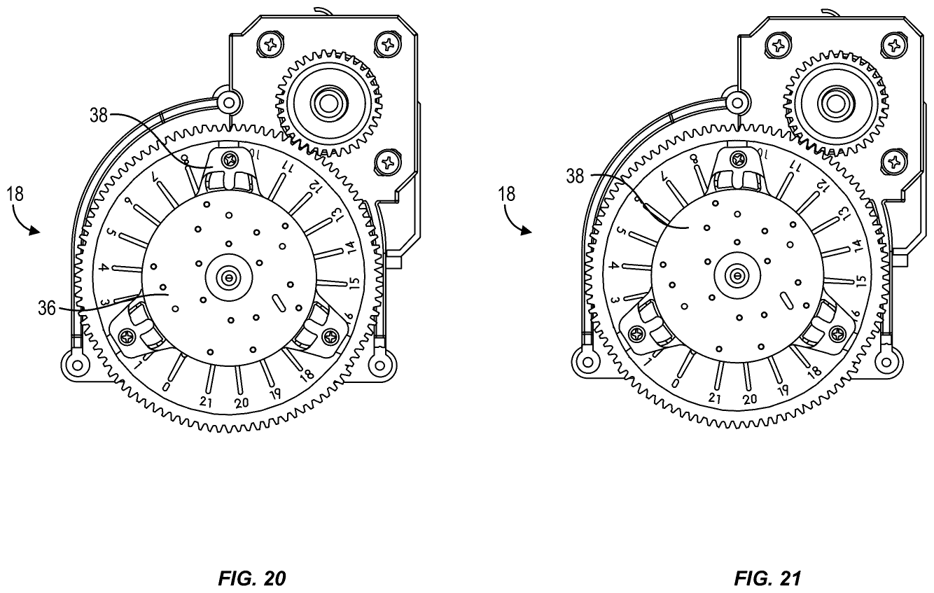

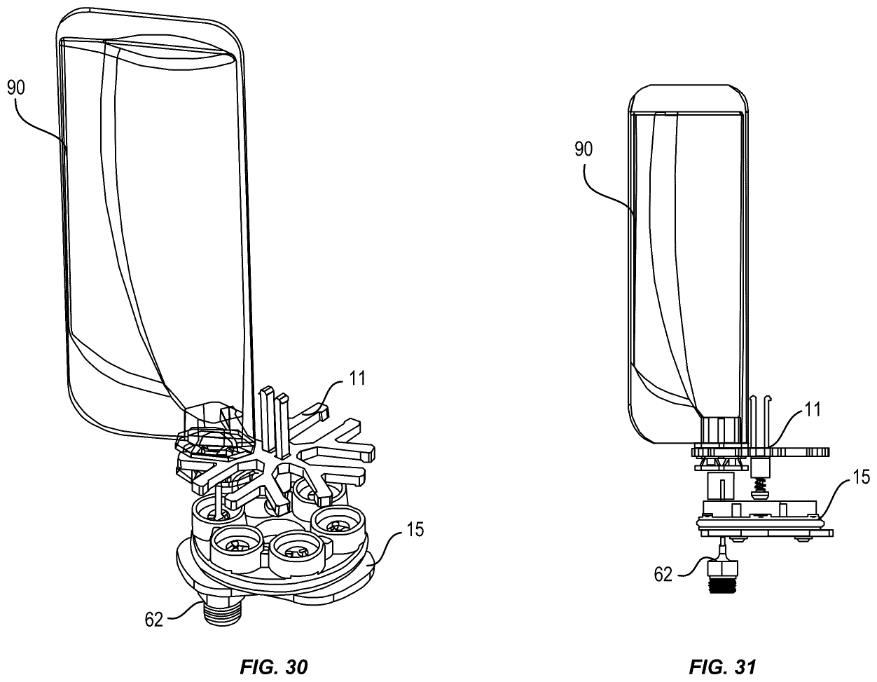

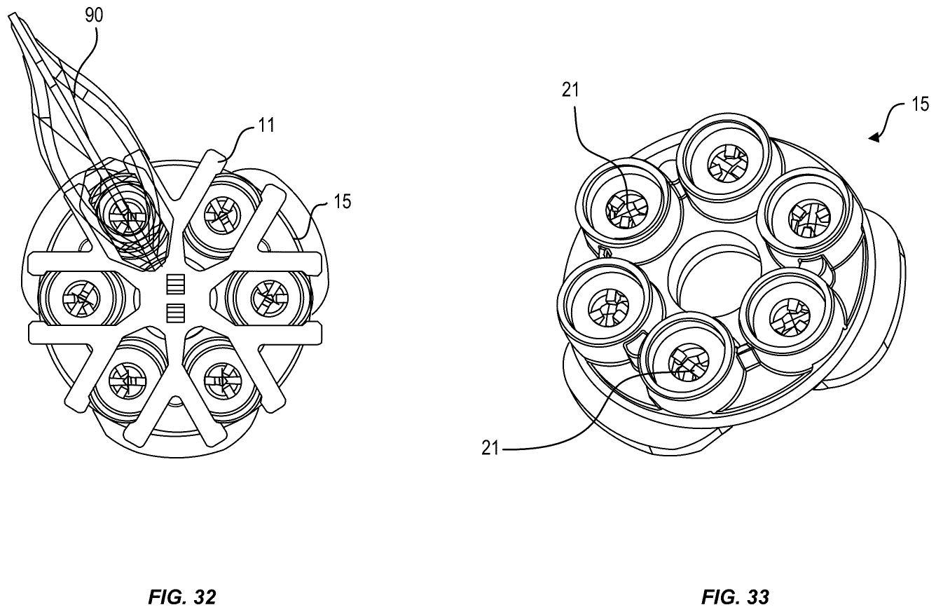

FIGS. 2-34 illustrate an example of the monitoring system 14 according to various embodiments. The monitoring system 14 generally includes a monitoring device 18 and a cartridge assembly 20 having one or more supplies of reagent. During a measurement cycle, and as discussed in detail below, reagent may be drawn from the cartridge assembly 20 into the monitoring device 18. Once drawn into the monitoring device 18, the reagent is mixed with water, and one or more water parameters are measured by the monitoring system 14.

### Monitoring Device

Referring to FIGS. 2-5, for example, the monitoring device 18 generally includes an interface assembly 22, a testing cell 24, a sensing system 26, and a double plunger assembly 28.

As illustrated in FIGS. 2-7 and 18-24, for example, the monitoring device 18 optionally includes a housing 30 for housing and/or supporting one or more components of the monitoring device 18. As non-limiting examples, the housing 30 may house and/or support the interface assembly 22, the testing cell 24, the sensing system 26, the double plunger assembly 28, one or more drive mechanisms 45, 75, the cartridge assembly 20, combinations thereof, and/or as otherwise desired. In certain embodiments, the housing 30 of the monitoring device 18 may include various features facilitating the positioning and/or installation of the monitoring system 14 inline with other equipment and/or at various locations relative to other equipment. As such, the particular configuration of the housing 30 should not be considered limiting.

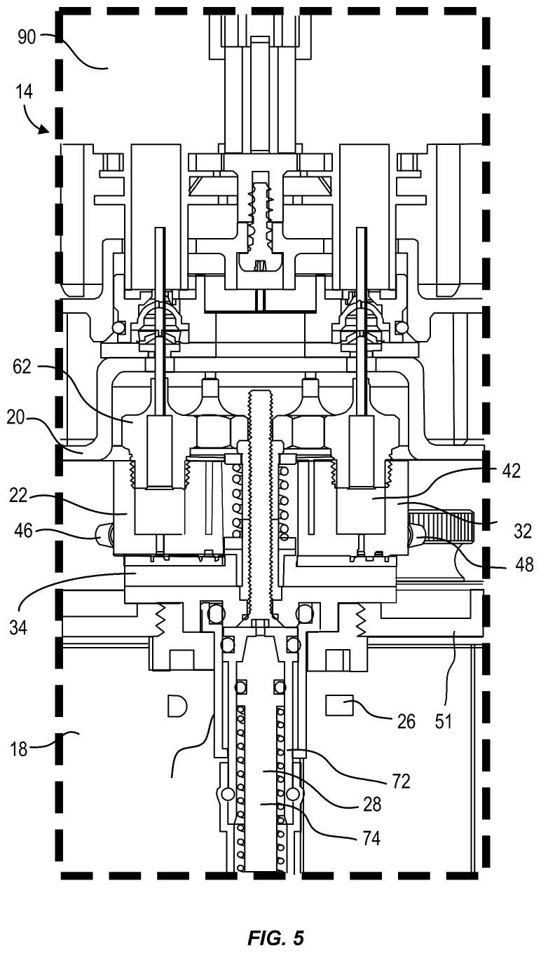

The sensing system 26 may be various devices and/or components suitable for measuring a water parameter as discussed in detail below. In some embodiments, and as illustrated in FIGS. 5 and 24-27 for example, the sensing system 26 includes one or more light sources 68 and one or more detectors 70. Additionally, or alternatively, the sensing system 26 may include a plurality of light sources, a plurality of detectors, and/or other suitable devices and/or combinations of devices as desired.

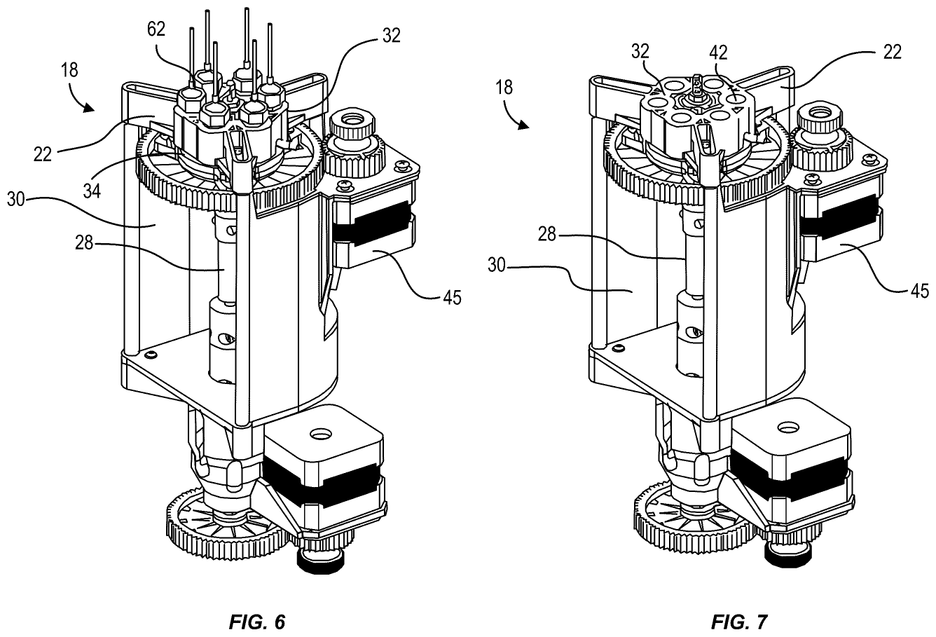

The interface assembly 22 of the monitoring device 18 may form the interface between the monitoring device 18 and the cartridge assembly 20. Additionally, or alternatively, the interface assembly 22 may form the interface between the monitoring system 14 and a flow of water from the pool system 10. As best illustrated in FIGS. 6-21, the interface assembly 22 of the monitoring device 18 generally includes an interface plate 32 and a valve assembly 34. The valve assembly 34 of the interface assembly 22 may include an upper plate 36 and a lower plate 38. In certain embodiments, and as discussed in detail below, the valve assembly 34 includes a valve gasket 40 between the interface plate 32 and the upper plate 36.

In certain embodiments, and as best illustrated in FIGS. 6-11 and 18, the interface plate 32 of the interface assembly 22 includes one or more reagent inflow ports 42. Each reagent flow port 42 may receive a flow of a corresponding reagent from the cartridge assembly 20 as described in detail below. While six reagent inflow ports 42 are illustrated in the interface plate 32, the number of reagent inflow ports 42 included on the interface plate 32 should not be considered limiting. As non-limiting examples, in other embodiments, an interface plate 32 may include one reagent inflow port 42, two reagent inflow ports 42, three reagent inflow ports 42, four reagent inflow ports 42, five reagent inflow ports 42, six reagent inflow ports 42, and/or more than six reagent inflow ports 42.

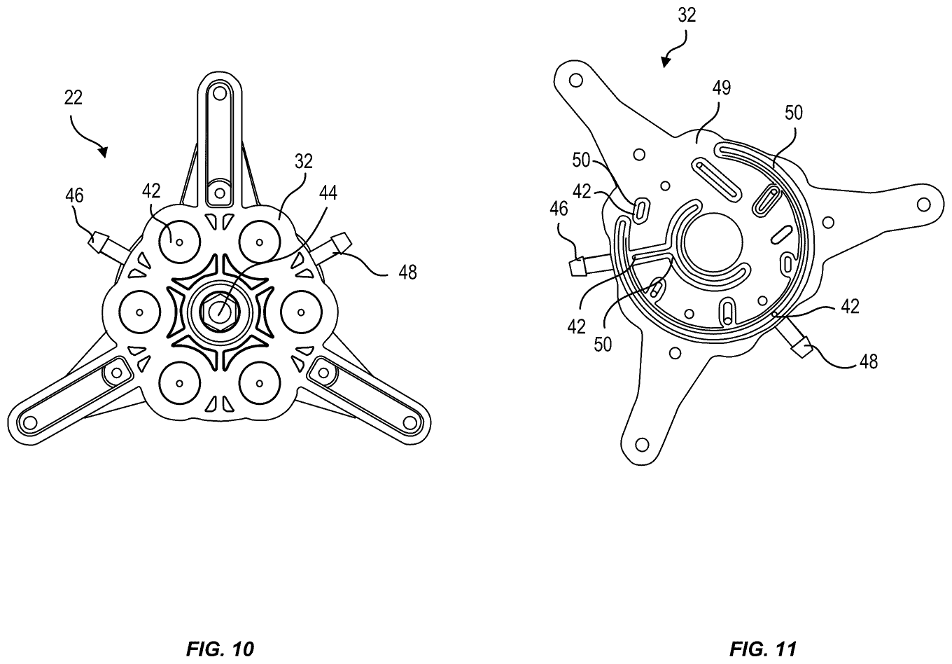

The reagent flow ports 42 may have various arrangements on the interface plate 32 as desired. Optionally, and as best illustrated in FIG. 10, the reagent inflow ports 42 may be arranged at a same distance from a center 44 of the interface plate 32. In such embodiments, the arrangement of reagent inflow ports 42 may promote engagement with the cartridge assembly having an optimized and/or compact arrangement of stores of reagent. However, in other embodiments, the reagent flow ports 42 need not be at a same radial distance.

In some embodiments, and as best illustrated in FIGS. 5, 6, and 28, for example, the reagent inflow ports 42 of the interface plate 32 may support, receive, and/or otherwise engage corresponding fluid connectors 62. The fluid connectors 62 may be various devices or mechanisms for engaging corresponding supplies of reagent of the cartridge assembly 20 as described in greater detail below. In the embodiment illustrated, the fluid connectors 62 are dispensing needles. However, in other embodiments, any type of fluid connector may be utilized as desired, such as but not limited to various dispensing tips, flow controllers, other dispensing needles, combinations thereof, and/or as otherwise desired. As such, the particular fluid connectors 62 illustrated should not be considered limiting.

Referring to FIGS. 5 and 8-11, for example, in addition to the reagent inflow ports 42, the interface plate 32 may include one or more water inflow ports 46 and one or more water outflow ports 48. In the embodiment illustrated, the interface plate 32 includes one water inflow port 46 and one water outflow port 48; however, the number of ports 46, 48 should not be considered limiting. In various embodiments, the water inflow port 46 of the interface plate 32 may be in fluid communication with the pool system 10 and receive a flow of water from the pool system 10. The water outflow port 48 may be in fluid communication with the pool system 10 and may be utilized for returning water and/or water mixed with reagent from the monitoring system 14 back to the pool system 10. The particular arrangement of the ports 42, 46, 48 on the interface plate 32 should not be considered limiting on the disclosure.

In various embodiments, and as best illustrated in FIG. 11, a lower surface 49 of the interface plate 32 includes a plurality of channels 50, and each channel 50 of the plurality of channels 50 corresponds and is in fluid communication with a particular one of the ports 42, 46, 48. As non-limiting examples, one channel 50 may be in fluid communication with the water inflow port 46, another channel 50 may be in fluid communication with the water outflow port 48, another channel 50 may be in fluid communication with a reagent inflow port 42 (e.g., for a pH reagent or other reagent), etc. In certain embodiments, a plurality of channels 50 may be in fluid communication with a corresponding reagent inflow port 42. As a non-limiting example, in embodiments with six reagent inflow ports 42, the interface plate 32 may include at least six channels 50, one for each of the reagent inflow ports 42.

As illustrated in FIG. 11, the channels 50 may extend in various patterns or shapes away from the particular port 42, 46, 48. The plurality of channels 50 corresponding to a particular port 42, 46, 48 and the arrangement of the channels 50 on the interface plate 32 may reduce and/or eliminate cross-contamination between the fluids (e.g., reagent, water, or mixed water) of the various ports. As non-limiting examples, a first reagent flowing from a first reagent inflow port 42 may flow through a first channel 50 and a second reagent flowing from a second reagent inflow port 42 may flow through a second channel 50 (i.e., without mixing or using the same channel 50 as the first reagent). The particular shapes or patterns of each channel 50 should not be considered limiting, and as illustrated, the channels 50 may extend radially, circumferentially, and/or as otherwise desired relative to the particular port 42, 46, 48 that it is in fluid communication with. The patterns and shapes of the channels 50 leading away from the particular port 42, 46, 48 may allow for ports of the valve assembly 34 to be in fluid communication with the ports 42, 46, 48 at locations other than the location of the ports 42, 46, 48.

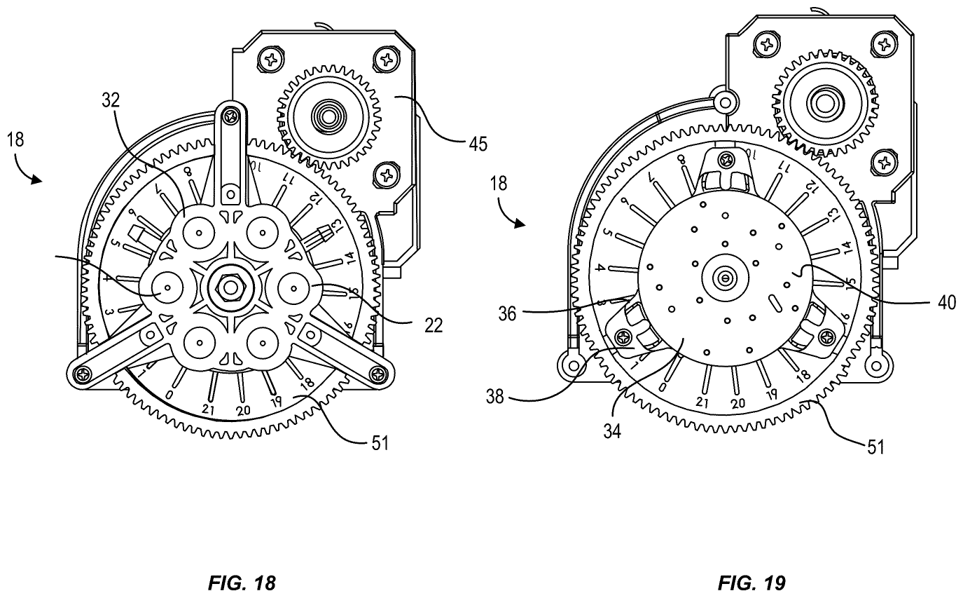

Referring to FIGS. 9 and 12-17, for example, the valve assembly 34 may be utilized for controlling water flow and reagent flow. In various embodiments, the valve assembly 34 is a ceramic valve assembly with one or more ceramic valves for controlling water flow and reagent flow. Optionally, the ceramic valve assembly as the valve assembly 34 is a multiport ceramic valve assembly. In some embodiments, the valve assembly 34 is a rotary valve assembly. In certain embodiments, the valve assembly 34 is a multiport rotary valve assembly, such as but not limited to a multiport ceramic rotary valve assembly. In these embodiments, and as discussed in detail below, the lower plate 38 is rotatable relative to the upper plate 36. In some embodiments, and as illustrated in FIGS. 18-21, for example, the lower plate 38 may be rotatable using a rotating member 51, and the lower plate 38 may be supported on the rotating member 51 using various devices or mechanisms as desired. In certain embodiments, the rotating member 51 may be rotated via one or more drive mechanisms 45 automatically, manually, and/or as otherwise desired. In other embodiments, the lower plate 38 may be rotatable and/or positionable relative to the upper plate 36 using other devices or mechanisms as desired.

As mentioned, the valve assembly 34 includes the upper plate 36, the lower plate 38, and optionally the valve gasket 40. Compared to traditional approaches which may require a plurality of valves, the valve assembly 34 provides the monitoring system 14 with a single valve controlling both the flow of water and the flow of reagents into the testing cell 24 while also minimizing and/or eliminating cross-contamination. The single valve used for introducing water and reagent into the testing cell 24 may further be utilized to expel water and reagent from the testing cell 24.

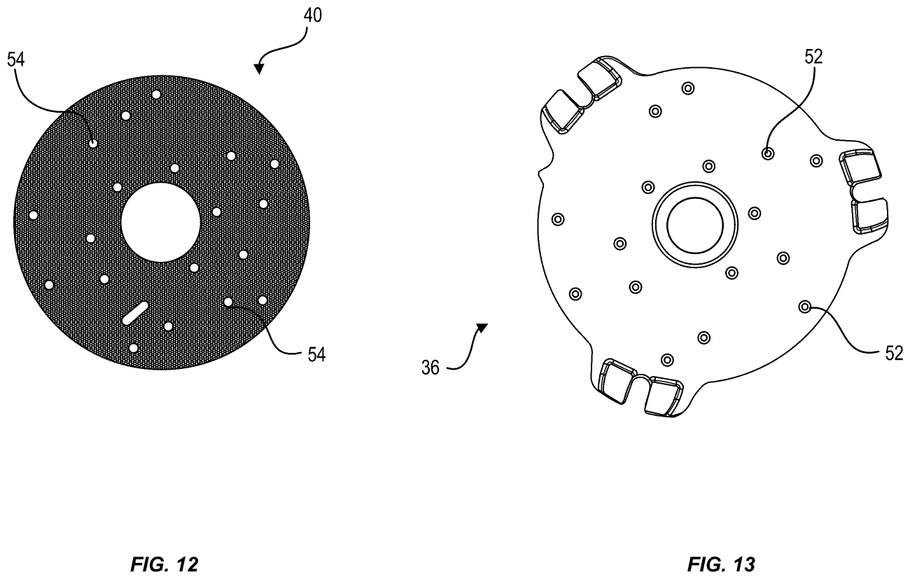

As best illustrated in FIGS. 12-14, the upper plate 36 of the valve assembly 34 includes one or more dispensing ports 52, each which corresponds with a particular one of the ports 42, 46, 48. In certain embodiments, the dispensing ports 52 are arranged to fluidly connect with a corresponding dispensing channel 50 at a location along the dispensing channel 50 other than the ports 42, 46, 48.

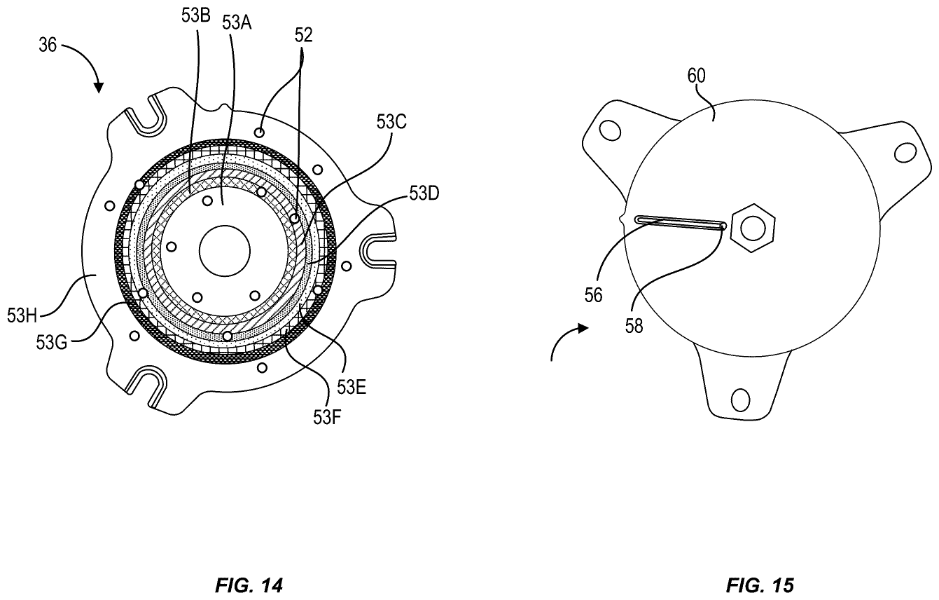

In various embodiments, and as best illustrated in FIG. 14, dispensing ports 52 for a particular one of the ports 42, 46, 48 may be arranged both radially and circumferentially on the upper plate 36. As a non-limiting example, and withe reference to FIG. 14, one or more dispensing ports 52 at a first radius (represented by circle 53A) may be in fluid communication with the water inflow port 46, one or more dispensing ports 52 at a second radius (represented by circle 53B) may be in fluid communication with a first reagent, one or more dispensing ports 52 at a third radius (represented by circle 53C) may be in fluid communication with a second reagent, one or more dispensing ports 52 at a fourth radius (represented by circle 53D) may be in fluid communication with a third reagent, one or more dispensing ports 52 at a fifth radius (represented by circle 53E) may be in fluid communication with a fourth reagent, one or more dispensing ports 52 at a sixth radius (represented by circle 53F) may be in fluid communication with a fifth reagent, one or more dispensing ports 52 at a seventh radius (represented by circle 53G) may be in fluid communication with a sixth reagent, and one or more dispensing ports 52 at an eighth radius (represented by circle 53H) may be in fluid communication with the water outflow port 48. The number and arrangement of ports 52 should not be considered limiting. In certain embodiments, the number of ports 52 on the upper plate 36 and the arrangement (radially and/or circumferentially) of the ports 52 on the upper plage 36 may at least partially depend upon the number of ports 42, 46, 48. In various embodiments, the radial and circumferential arrangement of the dispensing ports 52 may allow for accurate flow control of water and/or reagents while minimizing and/or eliminating cross-contamination.

Referring to FIG. 2, similar to the upper plate 36, the valve gasket 40 of the valve assembly 34 includes a plurality of dispensing ports 54. In certain embodiments, the number and the pattern or arrangement of the dispensing ports 54 may correspond at least partially with the number and pattern or arrangement of the dispensing ports 52 on the upper plate 36. When the interface assembly 22 is assembled, the valve gasket 40 may be provided between the upper plate 36 and the interface plate 32. The valve gasket 40 may be utilized to control the flow of fluid between the upper plate 36 and the interface plate 32 and minimize and/or eliminate leakage and/or cross-contamination of fluids.

Referring to FIGS. 15-17, the lower plate 38 of the valve assembly 34 includes a lower plate channel 56 and a lower plate port 58. In some embodiments, the lower plate channel 56 may be defined in an upper surface 60 of the lower plate 38. As illustrated in FIG. 15, the lower plate channel 56 may extend in a radial direction. However, in other embodiments, the lower plate channel 56 may have other arrangements as desired. In certain embodiments, the lower plate channel 56 is arranged or defined to intersect or move into alignment with one dispensing port 52 in use without intersecting or being in alignment with another dispensing port 52. In certain embodiments, due to the arrangement of the dispensing ports 52, the rotation of the lower plate 38 relative to the upper plate 36 may cause the lower plate channel 56 to rotate into alignment with one of the dispensing ports 52 without aligning with the other dispensing ports 52, thereby minimizing or preventing cross-contamination. As described in detail below and as illustrated in FIG. 26, for example, the lower plate port 58 may be in fluid communication with a testing chamber 64 of the testing cell 24. In such embodiments, the alignment of the lower plate channel 56 with one of the dispensing ports 52 may allow for fluid communication between a particular port 42, 46, 48 and the testing chamber 64 through the lower plate port 58.

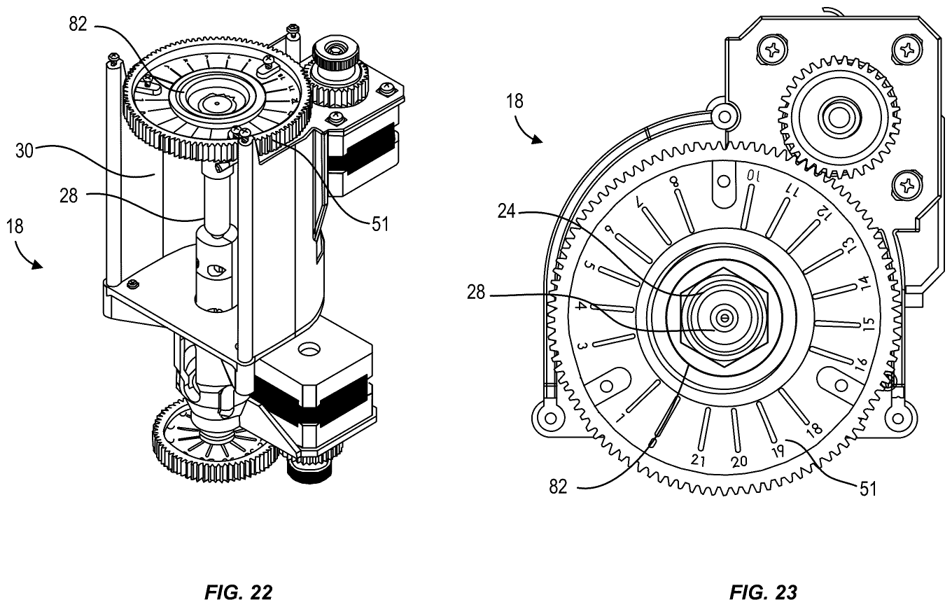

Referring to FIGS. 22-27, for example, in some embodiments, a holder 82 may support the testing cell 24 on the monitoring device 18, although in other embodiments, the holder 82 may be omitted. As such, the particular holder 82 illustrated should not be considered limiting.

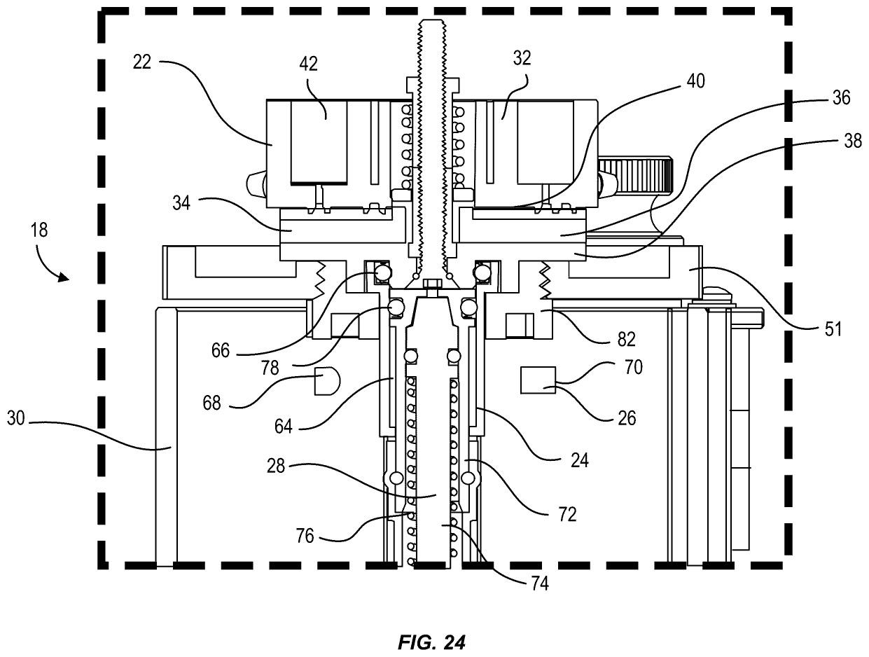

Referring to FIGS. 24-27, the interface assembly 22 may be arranged relative to the testing cell 24 such that the lower plate port 58 is in fluid communication with the testing chamber 64 of the testing cell 24. In certain embodiments, an upper sealing member 66 may seal the interface between the lower plate 38 and the upper sealing member 66, thereby sealing an upper end of the testing chamber 64. The testing chamber 64 itself has a fixed volume, and as discussed in detail below, mixing of water and reagent may be performed within the fixed volume. In various embodiments, the testing cell 24 may be constructed from various materials as desired and such that a water parameter of water within the testing chamber 64 may be measured by the sensing system 26.

As previously mentioned, the sensing system 26 may be various devices or combinations of devices as desired suitable for measuring a water parameter. In some embodiments, and as illustrated in FIGS. 24-27, for example, the sensing system 26 may include one or more light sources 68 and/or one or more detectors 70. As non-limiting examples, the sensing system 26 may be a photometer with a light source 68 and a detector 70 on opposing sides of the testing cell 24, and the detector 70 may measure the water parameter based on the light from the light source 68 which passes through water in the testing chamber 64. In another non-limiting example, a plurality of light sources 68 may be utilized, optionally at different wavelengths. As non-limiting examples, a first light source 68 may be provided at a first wavelength (e.g., 520 nm) and a second light source 68 may be provided at a second wavelength (e.g., 620 nm) to measure different water parameters. The number of light sources 68 and/or detectors 70 thus should not be considered limiting. In these embodiments where the sensing system 26 includes one or more light sources 68 and one or more detectors 70, the testing cell 24 may be a material that is transparent, semi-transparent, translucent, and/or semi-translucent to allow for the light from the light source 68 to pass through the testing chamber 64.

The double plunger assembly 28 of the monitoring device 18 may be utilized to draw water and reagent into the testing chamber 64, to mix the water and reagent in the testing chamber 64, and to dispel the water and reagent from the testing chamber 64. As best illustrated in FIGS. 24-27, the double plunger assembly 28 generally includes an outer plunger 72 and an inner plunger 74. In certain embodiments, the double plunger assembly 28 also includes a biasing member 76. In some embodiments, one or more components of the double plunger assembly 28 optionally may be positioned, directly or indirectly, using one or more actuators, such as but not limited to a drive mechanism 75 (see, e.g., FIGS. 2-4, 6, 7, and 22). In other embodiments, other actuators and/or drive mechanisms may be utilized as desired, and the drive mechanism 75 illustrated should not be considered limiting.

The outer plunger 72 may be positioned at least partially within the testing chamber 64 as illustrated in FIGS. 24-27, for example. In certain embodiments, a first lower sealing member 78 may seal the interface between the outer plunger 72 and the testing cell 24, thereby sealing the testing chamber 64 at a lower end. In certain embodiments, the outer plunger 72 defines an inner chamber 80, and an end 86 of the outer plunger 72 may define an aperture 88 providing access to the inner chamber 80 through the end 86.

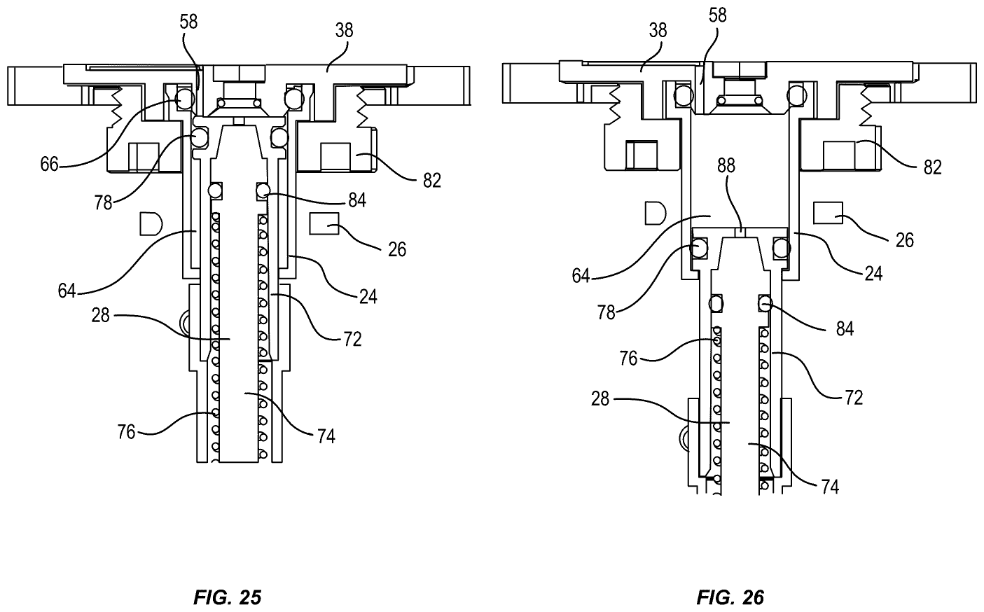

In various embodiments, and as illustrated in FIGS. 24-27, the inner plunger 74 may be positioned at least partially within the inner chamber 80 and such that the outer plunger 72 and the inner plunger 74 are axially aligned. In some embodiments, a second lower sealing member 84 may seal the interface between the outer plunger 72 and the inner plunger 74. When the biasing member 76 is included, the biasing member 76 may bias the inner plunger 74 toward the end 86 (FIGS. 25 and 26).

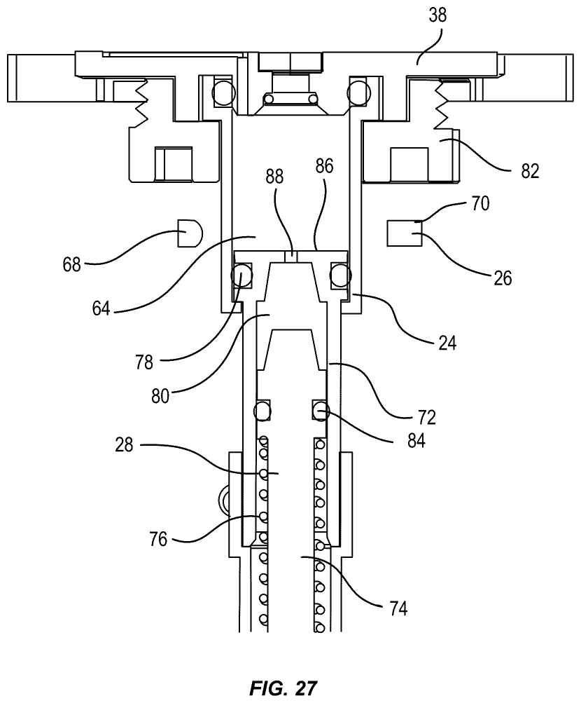

As illustrated in FIGS. 25 and 26, the outer plunger 72 may be movable relative to the testing cell 24 between an upper position (FIG. 25) and a lower position (FIG. 26) (and any intermediate position therebetween). The inner plunger 74 similarly may be movable relative to the outer plunger 72 between an upper position (FIGS. 25 and 26) and a lower position (FIG. 27) (and any intermediate position therebetween). In certain embodiments, the inner plunger 74 in the upper position may close or obstruct the aperture 88, thereby limiting or preventing access to the inner chamber 80.

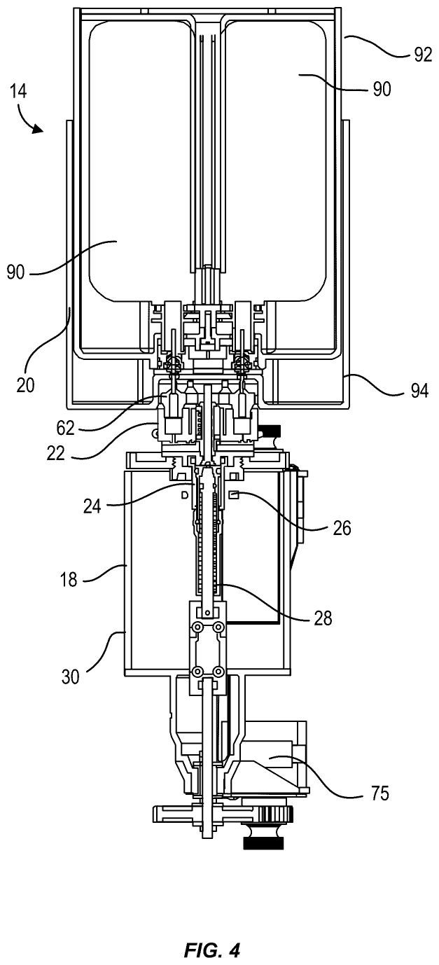

As mentioned, one or more drive mechanisms 75 (see FIG. 4) may be utilized to cause movement of the outer plunger 72 and/or the inner plunger 74. In certain embodiments, and as illustrated in FIG. 4, the outer plunger 72 optionally may be indirectly connected to the drive mechanism 75, and movement of the outer plunger 72 may be achieved by moving the inner plunger 74. In certain embodiments, and as discussed in detail below, movement of the outer plunger 72 from the upper position to the lower position relative to the testing cell 24 may be utilized to draw water into the testing chamber 64, and movement of the inner plunger 74 from the upper position to the lower position may be utilized to draw reagent into the testing chamber 64.

Optionally, the monitoring system 14 includes devices, components, and/or systems for receiving and/or providing data such as water parameter measurements to a user. As non-limiting examples, the monitoring system 14 may include a user interface on or associated with the monitoring device 18 and/or the cartridge assembly 20. Additionally, or alternatively, the monitoring device 18 and/or the cartridge assembly 20 may include a communication module enabling wired or wireless communication between the monitoring device 18 and/or the cartridge assembly 20 and a remote device, such as but not limited to a handheld device, user device, phone, computer, tablet, combinations thereof, and/or other remote devices as desired via various communication techniques.

### Cartridge Assembly

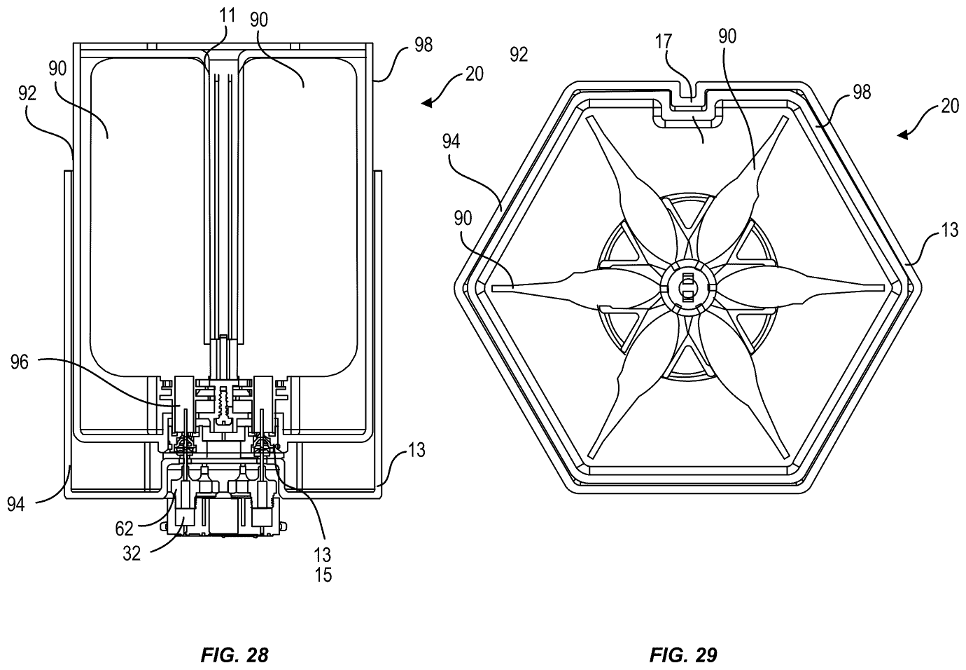

Referring to FIGS. 28-34, the cartridge assembly 20 of the monitoring system 14 generally includes one or more reagent holders 90 and a cartridge 92. Optionally, the cartridge assembly 20 may include a support 94, although it need not in other embodiments.

The reagent holders 90 may be various suitable devices or mechanisms suitable for holding a supply of reagent. In some non-limiting examples, the reagent holders 90 may be non-rigid material such as but not limited to a flexible pouch or package; however, in other embodiments, the reagent holders 90 may be other materials and/or constructions as desired. When a plurality of reagent holders 90 are included with the cartridge assembly 20, the reagent holders 90 may be a same type of reagent holder 90 or different types of reagent holders 90 as desired. Moreover, while six reagent holders 90 are illustrated in FIG. 29, the number of reagent holders 90 provided with the cartridge assembly 20 should not be considered limiting.

In certain embodiments, a port 96 of each reagent holder 90 may be initially sealed via various sealing mechanisms prior to engagement with the fluid connectors 62. Non-limiting examples of sealing mechanisms may include foils, barrier layers, tapes, films, plugs, stoppers, combinations thereof, and/or other suitable mechanisms as desired. The reagent provided within each reagent holder 90 may be various liquid reagents for measuring a parameter of water of the pool system 10. As non-limiting examples, a reagent holder 90 may hold reagent for measuring pH, free chlorine, total chlorine, total alkalinity, and/or cyanuric acid concentration.

Referring to FIGS. 28 and 29, the cartridge 92 of the cartridge assembly 20 includes a cartridge housing 98 for housing the reagent holders 90 therein. In certain embodiments, the cartridge 92 may optionally include one or more reagent holder supports 11 for supporting and/or positioning the reagent holders 90 relative to the cartridge 92. In certain embodiments, the cartridge 92 is removable from the support 94 such that the cartridge 92 can be replaced with a new cartridge having a new supply of reagent and/or such that the reagent holders 90 may be refilled as desired. In some embodiments, the cartridge 92 may be configured as a single unit such that every reagent holder 90 is installed or removed concurrently. In other embodiments, the cartridge 92 may be modular and allow for removal of individual reagent holders 90 from the support housing 13.

The support 94 of the cartridge assembly 20 includes an optional support housing 13 and a valve plate 15. In various embodiments, and as best illustrated in FIG. 29, the support housing 13 optionally includes an alignment feature 17, such as but not limited to a groove or channel, which may cooperate with a corresponding alignment feature 19 on the cartridge 92. In certain embodiments, the alignment features 17, 19 may be utilized to ensure proper positioning of the reagent holders 90 and may ensure that the reagents are aligned and in fluid communication with the proper ports of the monitoring device 18. As a non-limiting example, the alignment features 17, 19 may ensure that the reagent for measuring pH is properly aligned and in fluid communication with the ports of the monitoring device 18 for dispensing the reagent for measuring pH. Such alignment may further minimize and/or eliminate cross-contamination between different reagents used by the monitoring system 14. While a groove or channel is illustrated as alignment features 17, 19, other alignment features may be utilized as desired, and the particular alignment features illustrated should not be considered limiting.

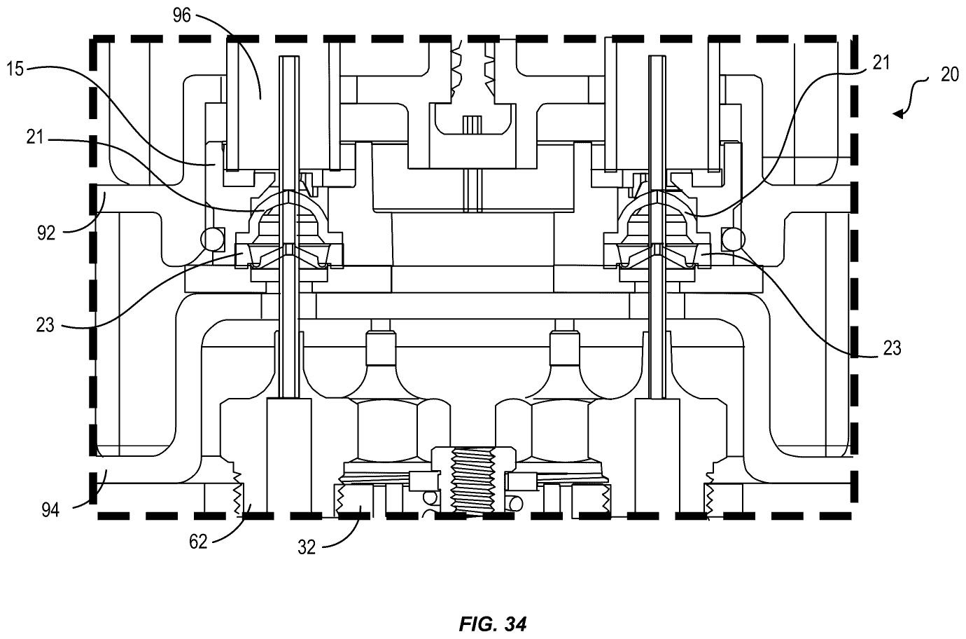

As best illustrated in FIGS. 33 and 34, the valve plate 15 may be utilized to control a flow of reagent from the reagent holders 90 to the fluid connectors 62 and subsequently to the monitoring device 18. Various devices or mechanisms may be utilized with the valve plate 15 to control the flow as desired. In some non-limiting examples, and as illustrated in FIGS. 33 and 34, the valve plate 15 optionally includes a dual valve system having a first valve 21 and a second valve 23 for each reagent holder 90. In these embodiments, the first valve 21 and the second valve 23 may be different types of valves. As a non-limiting example, the first valve 21 may be a dome valve and the second valve 23 may be a duckbill valve. In other embodiments, other valves, combinations of valves, and/or flow control devices or systems may be utilized as desired.

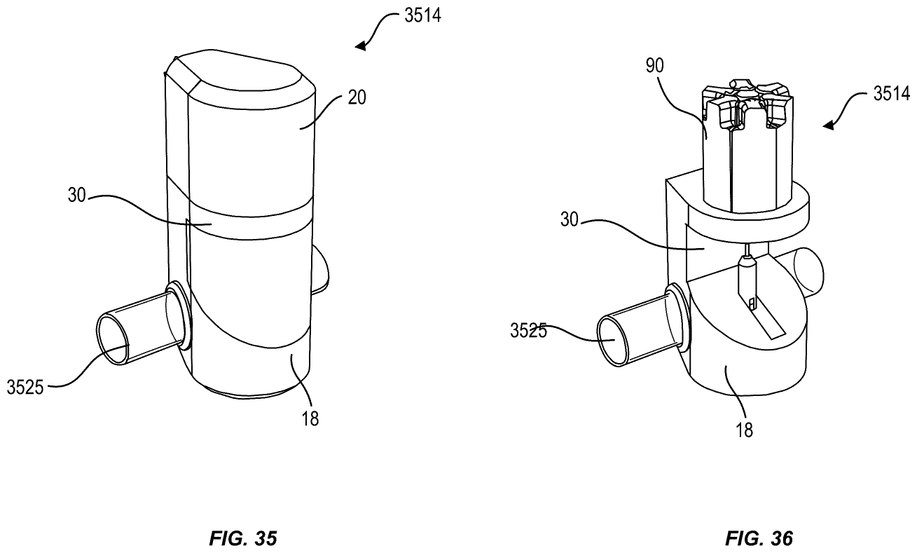

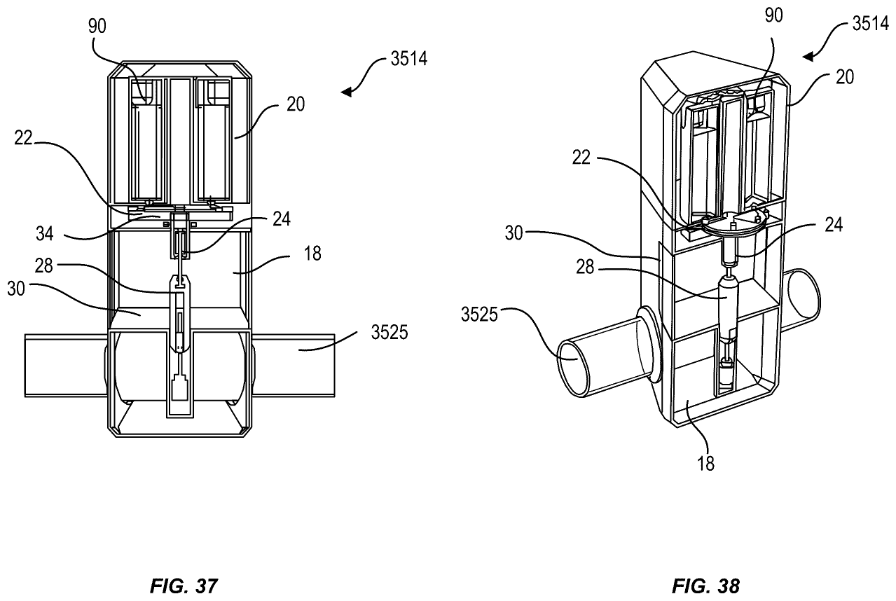

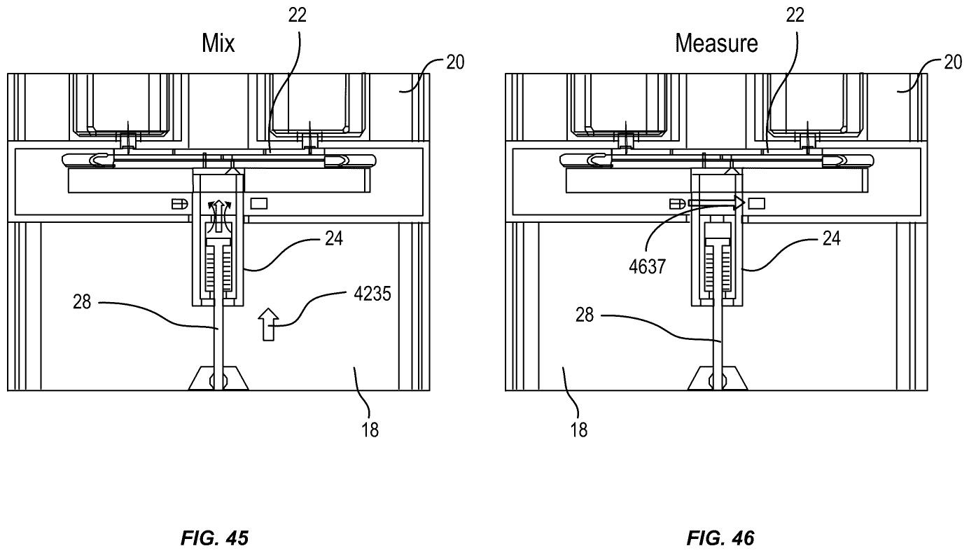

FIGS. 35-47 illustrate another example of a monitoring system 3514 for the pool system 10 according to embodiments. The monitoring system 3514 is similar to the monitoring system 14, and as illustrated in FIGS. 37 and 38, may be provided inline with conduit 3525 of the pool system 10. Compared to the monitoring system 14, the monitoring system 3514 omits the support 94 of the cartridge assembly 20. In addition, compared to the flexible reagent holders 90 of the monitoring system 14, the reagent holders 90 of the monitoring system 3514 are rigid. Similar to the monitoring system 14, among other components, the monitoring system 3514 includes the testing cell 24, the sensing system 26, the double plunger assembly 28, and the valve assembly 34.

Various other features and combinations of features may be utilized with the monitoring systems described herein, and the aforementioned advantages should not be considered limiting.

### Measurement Process

The monitoring systems described herein may allow for improved processing of water samples to obtain measurement(s) of one or more water parameters. A non-limiting example of a measurement process is described below in the context of one reagent, but the process may be repeated for each reagent and/or as many times as desired. In certain embodiments, a measurement cycle may include performing the measurement process for each reagent and/or to obtain a water parameter measurement of pH, free chlorine, total chlorine, total alkalinity, cyanuric acid concentration and/or other water parameter measurement. The following representative method makes reference to a measurement of pH, however the description should not be considered limiting and instead may be utilized for obtaining any water parameter measurement as desired.

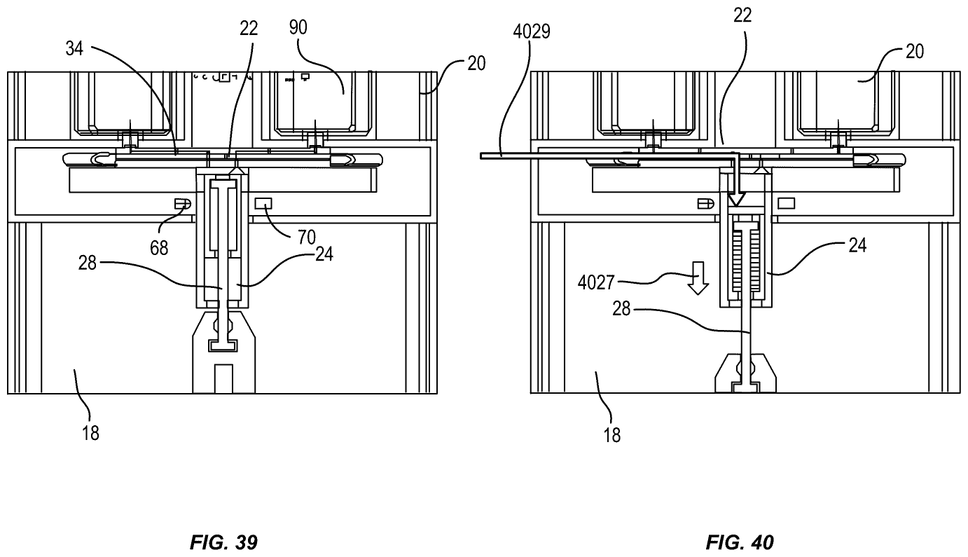

Referring to FIGS. 25 and 39, the measurement process for a water parameter (e.g., pH) may begin with the valve assembly 34 in a closed configuration. As illustrated in FIGS. 25 and 39, in certain embodiments, in the closed configuration, the outer plunger 72 and the inner plunger 74 may be in their upper positions, and the end 86 of the outer plunger 72 may abut the lower plate 38, further closing the lower plate port 58.

Referring to FIGS. 26 and 40, in a next stage of the measurement process, the valve assembly 34 is rotated (e.g., via drive mechanism 45) such that the lower plate port 58 is in fluid communication with the water inflow port 46. The outer plunger 72 then may be moved to the lower position (e.g., via drive mechanism 75) (represented by arrow 4027 in FIGS. 26 and 40), which may draw a volume of water into the testing chamber 64 (represented by arrow 4029 in FIG. 40).

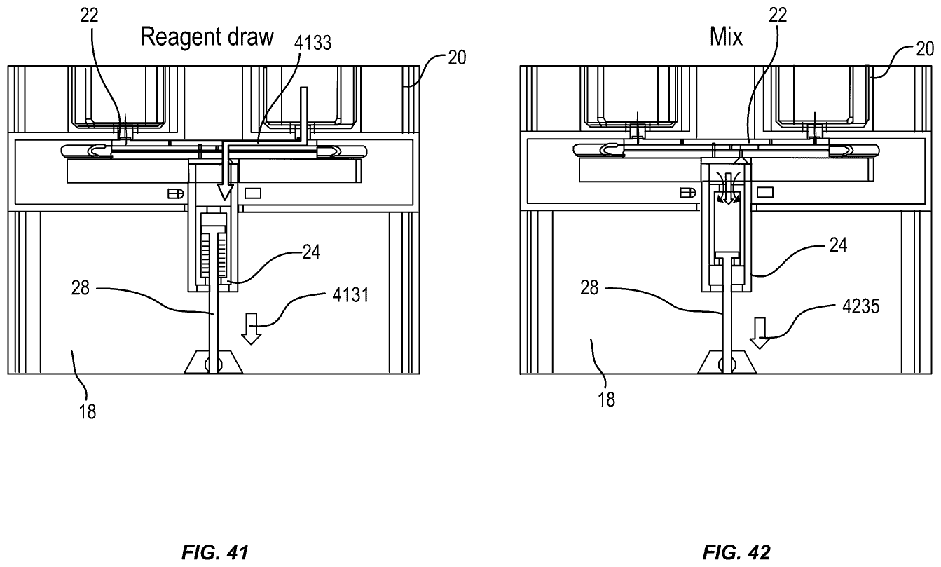

Referring to FIGS. 27 and 41, with the water in the testing chamber 64, the valve assembly 34 is rotated such that the lower plate port 58 is in fluid communication with the desired reagent inflow port 42 (e.g., the pH reagent inflow port). The inner plunger 74 may be moved to the lower position (represented by arrow 4131 in FIGS. 27 and 41), which draws a volume of the reagent (e.g., the pH reagent) into the testing chamber 64 (represented by arrow 4133 in FIG. 41). In certain embodiments, the volume of the reagent drawn into the testing chamber 64 is less than the volume of the water drawn into the testing chamber 64, although it need not be in other embodiments. As illustrated in FIGS. 27 and 41, the inner plunger 74 moving to its lower position in turn may partially draw water and/or reagent into the inner chamber 80. In certain embodiments, utilizing the inner chamber 80 to partially accommodate the water and the reagent may allow for a sufficient volume of water and reagent to be drawn into the testing cell 24 while maintaining the overall fixed volume of the testing chamber 64. In certain embodiments, the inner chamber 80 may allow for increasing the volume available for mixing and/or receiving the water and reagents while maintaining a position of the outer plunger 72.

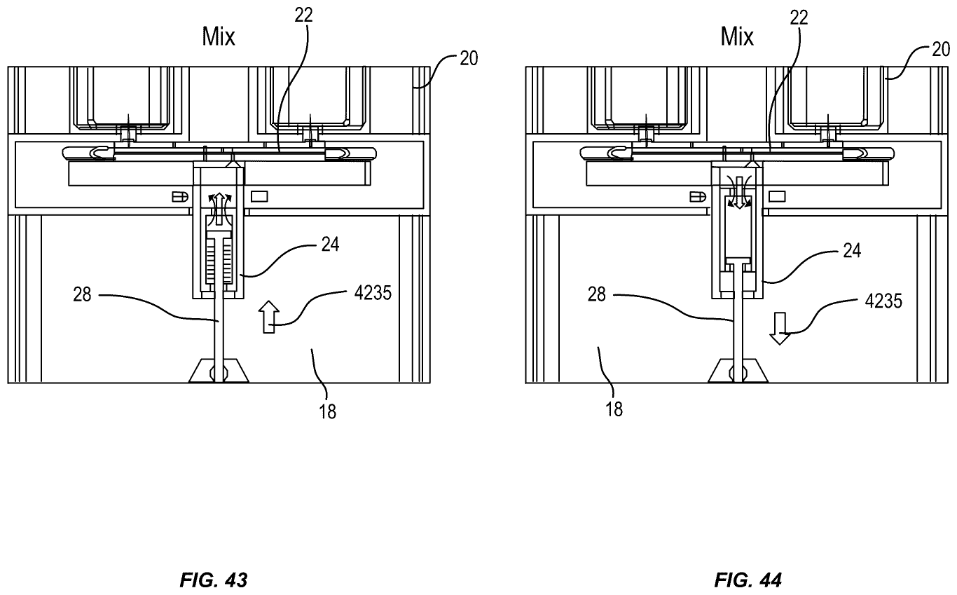

In various embodiments, after the reagent and water are drawn into the testing chamber 64, the valve assembly 34 may be rotated to a closed configuration. In certain embodiments, in the closed configuration, the valve assembly 34 may seal the testing chamber 64. Referring to FIGS. 42-45, with the valve assembly 34 in the closed configuration, the inner plunger 74 may be moved up and down (represented by arrows 4235 in FIGS. 42-45) to mix the water and the reagent. In certain optional embodiments, and as illustrated in FIGS. 42 and 43, because the testing chamber 64 is sealed, initial downward movement of the inner plunger 74 from its lower position to an extended position (see FIGS. 42 and 44) may cause upward movement of the outer plunger 72 due to pressure. In various embodiments, such relative movement between the plungers 72, 74 may further promote mixing of the water with the reagent.

Referring to FIG. 46, after mixing the water and the reagent, the inner plunger 74 may return to its lower position, and the measurement process includes measuring the mixed water using the sensing system 26 (represented by arrow 4637 in FIG. 46). The measurement obtained by the sensing system 26 may be provided to a control system (e.g., processor and/or memory) for further analysis or action, presented to a user on a user interface of the monitoring system, communicated to a remote location and/or a remote user device, and/or otherwise processed as desired. As a non-limiting example, based on the measurement obtained by the sensing system 26, the control system may automatically adjust and/or control a dispensing of chemicals into the pool (e.g., by controlling a chemical dosing system and/or as otherwise desired.

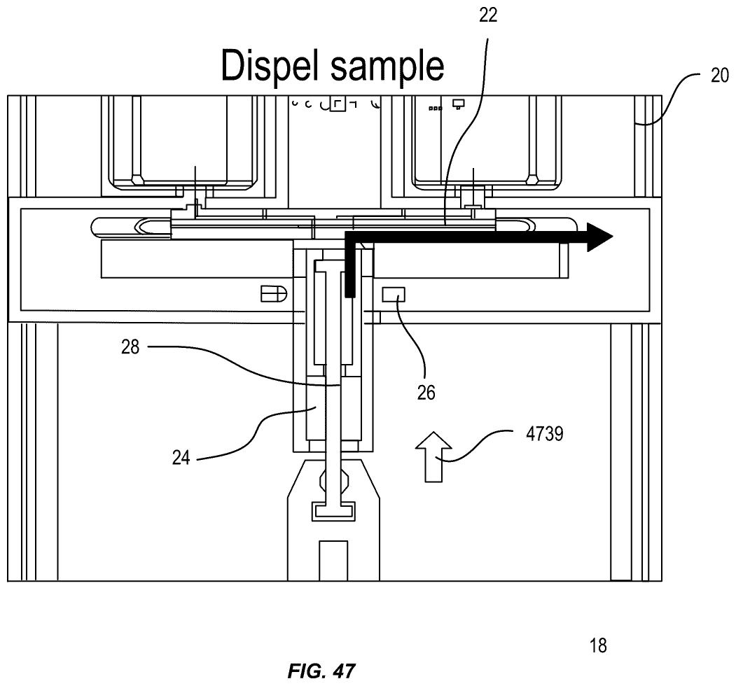

Referring to FIG. 47, after the measurement of the water is made, the valve assembly 34 may be rotated such that the lower plate port 58 is in fluid communication with the water outflow port 48, and the plungers 72, 74 are moved to their upper positions (represented by arrow 4739) to expel the mixed water and reagent from the testing chamber 64 back into the pool system 10.

The systems and methods described herein thus provide automatic, accurate, and reliable measurement of water parameters using liquid reagents on the pool pad and/or inline with other components of the pool system. In certain embodiments, the improved water measurements may be provided to a consumer or professional locally on the monitoring device itself and/or remotely as desired, such as but not limited to via an app. In certain embodiments, the inline and automatic water measurements may reduce and/or eliminate the need for physical involvement by a consumer or professional to physically go to the pool to collect the sample of water and manually perform the measurement or transport the water sample to a laboratory. The systems and methods described herein may provide accurate water measurements via liquid reagents, and further may be optimally placed to avoid low or no-flow locations, to avoid interaction with unwanted users, and/or to avoid other locations and/or environments that may compromise the accuracy of the water measurements and/or the condition of the device itself. Various other benefits and advantages may be realized with the systems, devices, and methods provided herein, and the aforementioned advantages should not be considered limiting.

Exemplary concepts or combinations of features of the invention may include:
A. A method of monitoring water quality of water of a swimming pool or spa, the method comprising mixing the water and a reagent in a fixed volume using a double plunger.
B. A system for monitoring water quality of a swimming pool or spa, the system comprising a double plunger and a fixed volume, wherein the double plunger is configured to mix water of the swimming pool or spa and a reagent within the fixed volume.
C. A system for monitoring water quality of a swimming pool or spa, the system comprising a double plunger and a multiport rotary valve.
D. A reagent based testing system with ceramic valves for controlling water and reagent flow.
E. A system for monitoring water quality of a swimming pool or spa, the system comprising a double plunger and a multiport ceramic valve.
F. A system for monitoring water quality of a swimming pool or spa, the system configured to measure water parameters using liquid reagents and a photometer, comprises a single valve for controlling a flow of water and the liquid reagents into a testing chamber.
G. A method of monitoring water quality of water of a swimming pool or spa, the method comprising receiving a flow of water from a circulation system of the swimming pool or spa and measuring one or more water parameters using liquid reagents and a photometer.
H. A system for monitoring water quality of a swimming pool or spa, the system comprising a multiport rotary valve with at least a first reagent dispensing port for a first reagent and a second reagent dispensing port for a second reagent, and wherein the first reagent dispensing port and the second reagent dispensing port are different distances from a center of the multiport rotary valve.
I. A system for monitoring water quality of a swimming pool or spa, the system comprising a multiport rotary valve assembly, wherein the multiport rotary valve assembly comprises a plurality of reagent inflow ports and a plurality of reagent dispensing ports, wherein the plurality of reagent inflow ports are a same distance from a center of the multiport rotary valve assembly, and wherein at least a first reagent dispensing port and a second reagent dispensing port of the plurality of reagent dispensing ports are different distances from the center of the multiport rotary valve assembly.
J. A system for monitoring water quality of a swimming pool or spa, the system comprising a double plunger, a testing chamber having a fixed volume, and a multiport rotary valve.
K. A system for monitoring water quality of a swimming pool or spa, the system comprising a testing cell defining a testing chamber and a single valve for controlling a flow of water and the liquid reagents into a testing chamber and for controlling a flow of water and liquid reagent from testing chamber.
L. A method of monitoring water quality of water of a swimming pool or spa, the method comprising:
   i. drawing water into a testing chamber using a double plunger;
   ii. rotating a multiport rotary valve to first position enabling fluid communication with a first reagent;
   iii. drawing the first reagent into the testing chamber using the double plunger;
   iv. mixing the first reagent and the water in the testing chamber using the double plunger; and
   v. measuring a water parameter of the mixed water and the first reagent in the testing chamber using a photometer.
M. The system or method of any preceding or subsequent statement or combination of statements, where the double plunger comprises a first plunger and a second plunger, wherein the first plunger is movable within the testing chamber, and wherein the second plunger is movable within the first plunger.
N. The system or method of any preceding or subsequent statement or combination of statements, wherein the first plunger is configured to draw a volume of water into the testing chamber, wherein the second plunger is configured to draw a volume of reagent into the testing chamber, and wherein the volume of water is greater than the volume of reagent.
O. The system or method of any preceding or subsequent statement or combination of statements, further comprising a cartridge assembly comprising at least one flexible package containing a supply of a reagent, wherein the system is configured to draw the reagent from the cartridge assembly.
P. The system or method of any preceding or subsequent statement or combination of statements, further comprising a multiport rotary valve.
Q. The system or method of any preceding or subsequent statement or combination of statements, wherein the multiport rotary valve is a multiport rotary ceramic valve.
R. The system or method of any preceding or subsequent statement or combination of statements, wherein the single valve is a ceramic valve.
S. The system or method of any preceding or subsequent statement or combination of statements, further comprising a photometer configured to measure one or more water parameters of the water and liquid reagent in the testing chamber.
T. The system or method of any preceding or subsequent statement or combination of statements, further comprising a testing chamber comprising a fixed volume; and a double plunger.

These examples are not intended to be mutually exclusive, exhaustive, or restrictive in any way, and the invention is not limited to these example embodiments but rather encompasses all possible modifications and variations within the scope of any claims ultimately drafted and issued in connection with the invention (and their equivalents). For avoidance of doubt, any combination of features not physically impossible or expressly identified as non-combinable herein may be within the scope of the invention. Further, although applicant has described devices and techniques for use principally with swimming pools or spas, persons skilled in the relevant field will recognize that the present invention conceivably could be employed in connection with other water containing vessels and in other manners, particularly but not limited to underwater installations. Finally, references to “pools” and “swimming pools” herein may also refer to spas or other water containing vessels used for recreation, training, or therapy.

That which is claimed:

1. A system for monitoring water quality of a swimming pool or spa, the system comprising a double plunger and a fixed volume, wherein the double plunger is configured to mix within the fixed volume (i) water of the swimming pool or spa and (ii) a reagent.

2. The system of claim 1, further comprising a multiport rotary valve.

3. The system of claim 2, wherein the multiport rotary valve is a multiport rotary ceramic valve.

4. The system of claim 2, wherein the multiport rotary valve comprises at least a first reagent dispensing port for a first reagent and a second reagent dispensing port for a second reagent, and wherein the first reagent dispensing port and the second reagent dispensing port are different distances from a center of the multiport rotary valve.

5. The system of claim 2, wherein the multiport rotary valve comprises a plurality of reagent inflow ports and a plurality of reagent dispensing ports, wherein the plurality of reagent inflow ports are a same distance from a center of the multiport rotary valve assembly, and wherein at least a first reagent dispensing port and a second reagent dispensing port of the plurality of reagent dispensing ports are different distances from the center of the multiport rotary valve assembly.

6. The system of claim 1, further comprising a photometer and a testing chamber, wherein the testing chamber defines the fixed volume, and wherein the photometer is configured to measure a water parameter of the water and reagent within the fixed volume.

7. The system of claim 1, wherein the double plunger comprises a first plunger and a second plunger, wherein the first plunger is movable within the fixed volume, and wherein the second plunger is movable within the first plunger.

8. The system of claim 1, wherein the double plunger comprises a first plunger and a second plunger, wherein the first plunger is configured to draw a volume of water into the fixed volume, wherein the second plunger is configured to draw a volume of reagent into the fixed volume, and wherein the volume of water is greater than the volume of reagent.

9. The system of claim 1, further comprising a cartridge assembly comprising at least one flexible package containing a supply of a reagent, wherein the system is configured to draw the reagent from the cartridge assembly.

10. A system for monitoring water quality of a swimming pool or spa, the system comprising:
a testing cell defining a testing chamber; and
a single valve for controlling a flow of water and a flow of liquid reagent into the testing chamber and for controlling a flow of water and liquid reagent from the testing chamber.

11. The system of claim 10, wherein the single valve is a ceramic valve.

12. The system of claim 10, further comprising a photometer configured to measure one or more water parameters of the water and liquid reagent in the testing chamber.

13. The system of claim 10, further comprising a double plunger, wherein the double plunger is configured to mix the water and liquid reagent within the testing chamber.

14. The system of claim 13, where the double plunger comprises a first plunger and a second plunger, wherein the first plunger is movable within the testing chamber, and wherein the second plunger is movable within the first plunger.

15. The system of claim 14, wherein the first plunger is configured to draw a volume of water into the testing chamber, wherein the second plunger is configured to draw a volume of reagent into the testing chamber, and wherein the volume of water is greater than the volume of reagent.

16. The system of claim 10, further comprising a cartridge assembly comprising at least one flexible package containing a supply of the liquid reagent, wherein the system is configured to draw the liquid reagent from the cartridge assembly.

17. A reagent-based testing system comprising a ceramic valve configured to control a flow of water and a flow of liquid reagent.

18. The reagent-based testing system of claim 17, wherein the ceramic valve is a multiport ceramic valve.

19. The reagent-based testing system of claim 17, wherein the ceramic valve comprises a plurality of reagent inflow ports and a plurality of reagent dispensing ports, wherein the plurality of reagent inflow ports are a same distance from a center, and wherein at least a first reagent dispensing port and a second reagent dispensing port of the plurality of reagent dispensing ports are different distances from the center.

20. The reagent-based testing system of claim 17, further comprising:
a testing chamber comprising a fixed volume; and
a double plunger.
